(19) United States
(12) Patent Application Publication
(10) Pub. No.: US 2021/0166798 A1
(43) Pub. Date: Jun. 3, 2021

(54) HEALTH MANAGEMENT DEVICE, HEALTH MANAGEMENT METHOD, AND NON-TRANSITORY RECORDING MEDIUM STORING PROGRAM

(71) Applicant: OMRON HEALTHCARE Co., Ltd., Kyoto (JP)

(72) Inventors: Tomohiro KUKITA, Kyoto (JP); Masahiko YUMOTO, Kyoto (JP); Tamio UEDA, Kyoto (JP)

(21) Appl. No.: 17/173,761

(22) Filed: Feb. 11, 2021

Related U.S. Application Data
(63) Continuation of application No. PCT/JP2019/029021, filed on Jul. 24, 2019.

(30) Foreign Application Priority Data
Aug. 20, 2018 (JP) 2018-154187

(51) Int. Cl. G16H 20/10

(52) U.S. Cl. CPC G16H 20/10

(57) ABSTRACT

A health management device according to an aspect includes a first determination unit configured to determine whether or not an indicator relating to a user has changed in a first time period toward achieving a preset goal, a second determination unit configured to determine whether or not the user has performed a behavior in a manner that satisfies a preset condition, in a second time period, which includes at least a portion of the first time period, and an information presentation unit configured to present information that facilitates achieving the goal in a case in which the first determination unit determines that the indicator has not changed in the first time period toward achieving the goal and the second determination unit determines that the user has performed the behavior in the second time period in a manner that satisfies the condition.

### CROSS-REFERENCE TO RELATED APPLICATIONS

This application is the U.S. national stage application filed pursuant to 35 U.S.C. 365(c) and 120 as a continuation of International Patent Application No. PCT/JP2019/029021, filed Jul. 24, 2019, which application claims priority from Japanese Patent Application No. 2018-154187, filed Aug. 20, 2018, which applications are incorporated herein by reference in their entireties.

### TECHNICAL FIELD

The present invention relates to, for example, technology for managing the health of a user.

### BACKGROUND ART

A known system provides advice to a user to help them achieve set goals relating to the health of the user. For example, in Patent Document 1 an advice generation system is described that determines motivation for achieving a goal on the basis of achievement state information indicative of the achievement state of the goal to be achieved by a user and action amount information indicative of the cumulative amount of action taken by the user to achieve the goal, and generates advice for the user on the basis of the determination result of the motivation.

### CITATION LIST

### Patent Literature

Patent Document 1: JP 2016-106698 A

### SUMMARY OF INVENTION

### Technical Problem

However, in the advice generation system described in Patent Document 1, appropriate advice cannot be provided to the user for the following reason. That is, the trend of change in the achievement state information is determined on the basis of a comparison between the achievement state at the start of a check period and the achievement state at the end of the check period, and does not take into account the achievement state during the check period. Thus, the generated achievement state information may not match the actual situation due to the achievement state changing temporarily just before or after the start or end of the check period. As a result, inappropriate advice may be provided to the user.

The present invention has been made in light of the circumstances described above, and an object of the present invention is to provide a health management device, a health management method, and a non-transitory recording medium storing a program capable of providing appropriate advice to a user for achieving a set goal relating to the health of the user.

### Solution to Problem

The present invention adopts the following configurations in order to solve the above problems.

A health management device according to an aspect includes, a measurement data acquisition unit configured to acquire measurement data output from a measurement device configured to measure an indicator relating to biological information of a user, a first determination unit configured to determine, on the basis of the measurement data whether or not the indicator has changed in a first time period toward achieving a preset goal, a behavior data acquisition unit configured to generate behavior data relating to behavior of the user on the basis of the measurement data, a second determination unit configured to determine, on the basis of the behavior data, whether or not the user has performed the behavior in a second time period in a manner that satisfies a preset condition, the second time period including at least a portion of the first time period, and an information presentation unit configured to present information that facilitates achieving the goal in a case in which the first determination unit determines that the indicator has not changed in the first time period toward achieving the goal and the second determination unit determines that the user has performed the behavior in the second time period in a manner that satisfies the condition.

According to the configuration described above, whether or not the cause of the indicator not changing toward achieving the goal is in the behavior of a target to be measured can be determined. This allows appropriate information that facilitates achieving the goal to be presented to the user. In other words, appropriate advice for achieving a set goal relating to the health of the user can be provided to the user.

In an aspect, the second time period may further include a day prior to the first time period. The indicator in the initial portion of the first time period is affected by the behavior of the user in the time period prior to the first time period. By the second time period including the day prior to the first time period, whether or not the cause of the indicator not changing in the first time period toward achieving the goal is in the behavior can be determined with greater accuracy.

In an aspect, the first determination unit may calculate an average value of the indicator for the first time period on the basis of the measurement data and determine, on the basis of a comparison between the average value being calculated and a threshold that is preset whether or not the indicator has changed in the first time period toward achieving the goal.

According to the configuration described above, the measurement result of the indicator in the entire first time period is considered in the determination. This allows a determination result based on the actual change of the indicator in the first time period to be obtained.

In an aspect, the first determination unit may calculate, on the basis of the measurement data, a number of days that the indicator was greater or less than a preset first threshold, in the first time period, and determines, on the basis of a comparison between the calculated number of days and a preset second threshold, whether or not the indicator has changed in the first time period toward achieving the goal.

According to the configuration described above, the measurement result of the indicator in the entire first time period is considered in the determination. This allows a determination result based on the actual change of the indicator in the first time period to be obtained.

In an aspect, the indicator may be blood pressure, and the behavior may be use of medication.

The above configuration allows the user to be provided with advice when blood pressure has not changed toward achieving the goal even though the user has appropriately used the medication.

In an aspect, the measurement device may be a blood pressure monitor configured to measure blood pressure per heartbeat, and the behavior data acquisition unit may generate the behavior data on the basis of the measurement data output from the blood pressure monitor.

According to the configuration described above, a determination is made by the second determination unit on the basis of measurement data relating to blood pressure. In this way, the user does not need to input the behavior data, improving user convenience.

### Advantageous Effects of Invention

According to the present invention, a health management device, a health management method, and a non-transitory recording medium storing a program capable of providing appropriate advice to a user for achieving a set goal relating to the health of the user.

### BRIEF DESCRIPTION OF DRAWINGS

FIG. 1 is a diagram illustrating a health management system according to an embodiment.

FIG. 2 is a block diagram illustrating a hardware configuration of a terminal device according to an embodiment.

FIG. 3 is a block diagram illustrating a software configuration of the terminal device illustrated in FIG. 2.

FIG. 4 is a diagram for describing the determination processing by a first determination unit and a second determination unit illustrated in FIG. 3.

FIG. 5 is a flowchart illustrating a health management method executed by the terminal device illustrated in FIGS. 2 and 3.

### DESCRIPTION OF EMBODIMENTS

Hereinafter, an embodiment of the present invention will be described with reference to the drawings.

### Application Example

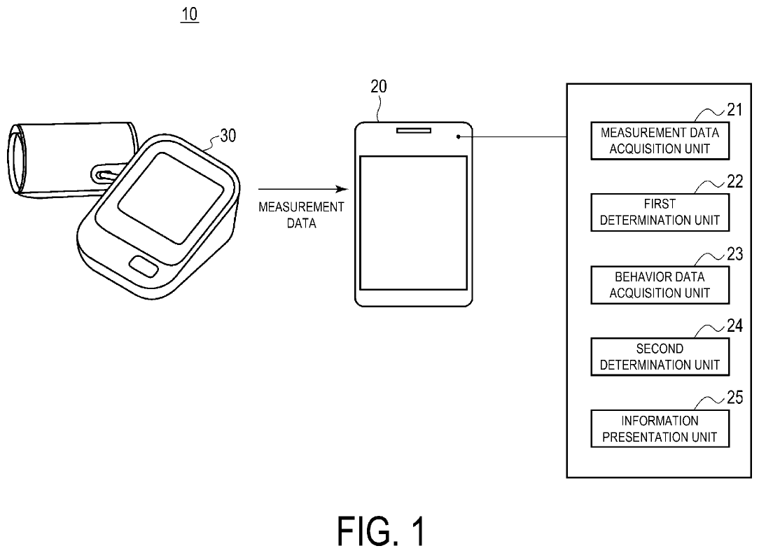

With reference to FIG. 1, an example of a case to which the present invention is applied will be described. FIG. 1 illustrates a health management system 10 according to an embodiment. In the example of FIG. 1, the health management system 10 includes a terminal device 20 and a blood pressure monitor 30. The terminal device 20 manages the health of the user in terms of blood pressure and corresponds to the “health management device” of the present invention. Blood pressure is an example of an indicator for a user. The blood pressure monitor 30 corresponds to the “measurement device” of the present invention.

The blood pressure monitor 30 is, for example, an oscillometric blood pressure monitor. The blood pressure monitor 30 measures the user's blood pressure and generates measurement data. The measurement data includes the systolic blood pressure (SBP) and the diastolic blood pressure (DBP), but is not limited thereto.

The terminal device 20 may be a computer such as a smart phone, a tablet personal computer (PC), a notebook PC, and a desktop PC, for example. The terminal device 20 includes a measurement data acquisition unit 21, a first determination unit 22, a behavior data acquisition unit 23, a second determination unit 24, and an information presentation unit 25.

The measurement data acquisition unit 21 acquires measurement data output from the blood pressure monitor 30. In one example, the measurement data is displayed on the blood pressure monitor 30 just after the completion of blood pressure measurement, and the user inputs the measurement data into the terminal device 20. In another example, the terminal device 20 wirelessly communicates with the blood pressure monitor 30 and receives the measurement data from the blood pressure monitor 30. For example, the user measures blood pressure with the blood pressure monitor 30 after waking up each day, so that the blood pressure measurements of each morning are recorded in the terminal device 20.

The first determination unit 22 determines, on the basis of the measurement data acquired by the measurement data acquisition unit 21, whether or not the blood pressure of the user has changed, in a first time period, toward achieving a preset goal relating to health. For example, in a case in which the user has hypertension, the goal to the user is to lower the blood pressure to a target value. The target value may be a value specified by the user him or herself, or may be a value specified by another person such as a physician. For example, the first determination unit 22 determines a trend in the change of the blood pressure on the basis of the blood pressure measurement values for each day in the first time period. In the case in which the blood pressure shows a declining trend, the first determination unit 22 determines that the blood pressure has changed, in the first time period, toward achieving the goal, and in the case in which the blood pressure does not show a declining trend, the first determination unit 22 determines that the blood pressure has not changed, in the first time period, toward achieving the goal.

The behavior data acquisition unit 23 acquires behavior data related to the medication of the user. The behavior data represents, for example, the history of the user taking an antihypertensive agent. For example, in the case in which the user has been instructed by a physician to take the antihypertensive agent every morning after breakfast, the behavior data includes information indicative of whether or not the user took the antihypertensive agent after breakfast each day. For example, when the user takes an antihypertensive agent after breakfast, the user inputs into the terminal device 20 that the user took the antihypertensive agent.

The second determination unit 24 determines, on the basis of the behavior data acquired by the behavior data acquisition unit 23, whether or not the medication has been taken in a second time period in a manner that satisfies a preset condition. The second time period includes the day prior to the first time period and at least a portion of the first time period. For example, the second determination unit 24 determines that the user has taken the medication in the second time period in a manner that satisfies the condition, when the medication adherence in the second time period is greater than a preset threshold. The medication adherence is the ratio at which the user took the medication (in this example, the antihypertensive agent) as per the instructions from the physician. In other words, the medication adherence is the ratio of the number of times the medication was actually taken to the number of times the medication should have been taken.

The information presentation unit 25 presents to the user information that facilitates achieving the goal when the first determination unit 22 determines that the blood pressure has not changed in the first time period toward achieving the goal and the second determination unit 24 determines that the user took the medication in the second time period in a manner that satisfies the condition. The conceivable cause of the blood pressure not decreasing to the target value despite the user taking the antihypertensive agent in an appropriate manner may be in the behavior of the user in terms of factors other than the medication. One plausible factor is high salt intake, for example. The information presentation unit 25 displays on a display device of the terminal device 20 a message saying “Please watch your salt intake when eating meals”, for example.

According to the terminal device 20 with the configuration described above, whether or not the cause of the blood pressure not changing in the first time period toward achieving the goal is the taking of the antihypertensive agent in the second time period can be determined. Information that facilitates achieving the goal is presented to the user when the user has appropriately taken the antihypertensive agent in the second time period but the blood pressure has not changed in the first time period toward achieving the goal. This allows appropriate information that facilitates achieving the goal to be presented to the user. In other words, appropriate advice for achieving a set goal relating to the health of the user can be provided to the user.

Typically, the user measures blood pressure after waking up and then eats breakfast and takes the antihypertensive agent. In this case, the blood pressure measurement value depends largely on whether or not the user took the antihypertensive agent the day before. Thus, the change in blood pressure in the first time period is affected not only by taking the medication in the first time period, but also by taking the medication in a time period prior to the first time period. Thus, by the second time period including the day prior to the first time period, whether or not the cause of the blood pressure not changing in the first time period toward achieving the goal is the taking of the antihypertensive agent in the second time period can be determined with greater accuracy.

Next, a health management device according to an embodiment will be described in further detail. In the embodiment/s described below, the health management device wirelessly communicates with the measurement device (for example, a blood pressure monitor).

### Configuration Example

### Hardware Configuration

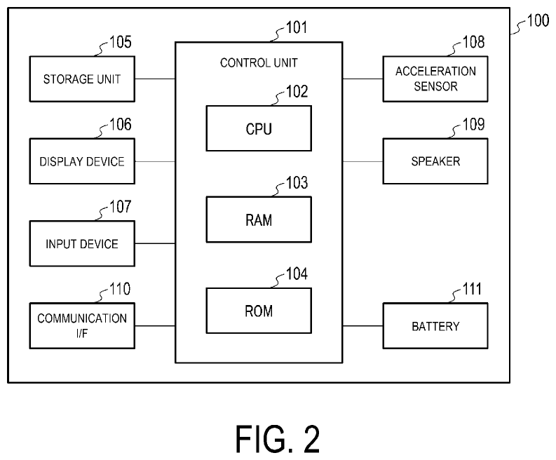

An example of the hardware configuration of a terminal device 100, i.e., a health management device, according to an embodiment will be described with reference to FIG. 2. FIG. 2 illustrates an example of the hardware configuration of the terminal device 100. In the example of FIG. 2, the terminal device 100 is a mobile device such as a smart phone and includes a control unit 101, a storage unit 105, a display device 106, an input device 107, an acceleration sensor 108, a speaker 109, a communication interface 110, and a battery 111.

The control unit 101 includes a central processing unit (CPU) 102, a random access memory (RAM) 103, a read only memory (ROM) 104, and the like and controls each component. For example, the storage unit 105 is an auxiliary storage device, such as a hard disk drive (HDD), a semiconductor memory (for example, a flash memory), and stores, in a non-volatile manner, programs executed by the control unit 101, settings data necessary for executing the programs, measurement data, and the like. A storage medium included in the storage unit 105 is a medium that stores information such as a program by electrical, magnetic, optical, mechanical, or chemical action so that a computer, a machine, or the like can read the information such as the program being recorded. Note that at least one or all of the programs may be stored in the ROM 104.

The display device 106 is controlled by the control unit 101 and displays information such as information that facilitates achieving a goal. The display device 106 may be an organic electroluminescence (EL) display, for example. Organic EL displays are sometimes referred to as organic light emitting diode (OLED) displays. Also, the display device 106 may be a display device of a different type, such as a liquid crystal display (LCD) device, for example.

The input device 107 allows the user to input an instruction to the terminal device 100. The input device 107 accepts an operation by a user, for example, an operation indicating that an antihypertensive agent has been taken. The input device 107 typically includes a touch panel provided on the screen of the display device 106. The input device 107 may further include one or more hardware buttons.

The acceleration sensor 108 is, for example, a triaxial acceleration sensor and outputs an acceleration signal representing acceleration in three directions orthogonal to each other. The speaker 109 is supplied with an audio signal from the control unit 101 and converts the audio signal into sound.

The communication interface 110 is an interface for communicating with an external communication device. The communication interface 110 includes, for example, a short-range wireless communication module, such as a Bluetooth (trade name) module or a Bluetooth Low Energy (BLE) module and communicates directly with the communication device (for example, a blood pressure monitor). The communication interface 110 typically includes a wireless module such as a wireless local area network (LAN) module instead of or in addition to the short-range wireless communication module. The communication interface 110 communicates with a communication device (for example, a server) via a communication network such as the Internet using a Bluetooth module or a wireless LAN module. The communication interface 110 may also include a terminal such as a micro Universal Serial Bus (USB) connector to communicate with the communication device in a wired manner.

The battery 111 supplies power to each component in the terminal device 100. The battery 111 is a rechargeable battery, for example.

Note that, with regard to the specific hardware configuration of the terminal device 100, a component can be omitted, replaced, and added as appropriate according to the embodiment. For example, the control unit 101 may include a plurality of processors.

### Software Configuration

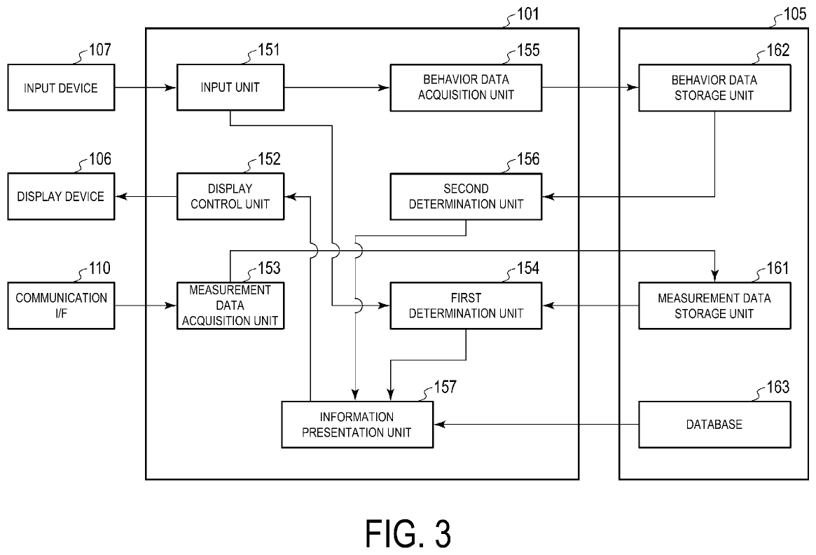

An example of the software configuration of the terminal device 100 will be described with reference to FIG. 3. In the example of FIG. 3, the terminal device 100 includes an input unit 151, a display control unit 152, a measurement data acquisition unit 153, a first determination unit 154, a behavior data acquisition unit 155, a second determination unit 156, an information presentation unit 157, a measurement data storage unit 161, a behavior data storage unit 162, and a database 163. The input unit 151, the display control unit 152, the measurement data acquisition unit 153, the first determination unit 154, the behavior data acquisition unit 155, the second determination unit 156, and the information presentation unit 157 executes the following processing by the control unit 101 of the terminal device 100 executing a program stored in the storage unit 105. When the control unit 101 executes the program, the control unit 101 deploys the program in the RAM 103. Then, the control unit 101 causes the CPU 102 to interpret and execute the program deployed in the RAM 103 to control each of the components. The measurement data storage unit 161, the behavior data storage unit 162, and the database 163 are provided in the storage unit 105.

The input unit 151 accepts input from the user. The input unit 151 receives, from the input device 107, an operation signal corresponding to the operation performed by the user on the input device 107 and determines the contents of the instruction input by the user on the basis of the operation signal. For example, when the user uses the input device 107 to input goal information, which is information relating to a goal, the input unit 151 sends the input goal information to the first determination unit 154. Accordingly, the goal of the user is set at the first determination unit 154. Furthermore, when the user uses the input device 107 to input that the antihypertensive agent has been taken, the input unit 151 sends, to the behavior data acquisition unit 155, a signal indicating that the user has taken the antihypertensive agent.

The display control unit 152 controls the display device 106 and causes information to be displayed on the display device 106. For example, the display control unit 152 causes the display device 106 to display a screen for setting a goal. Also, the display control unit 152 receives information to be presented to the user from the information presentation unit 157 and causes the display device 106 to display this information.

The measurement data acquisition unit 153 acquires measurement data generated by the blood pressure monitor and stores the measurement data in the measurement data storage unit 161. Specifically, the measurement data acquisition unit 153 receives measurement data from the blood pressure monitor via the communication interface 110. For example, the user uses the blood pressure monitor to measure blood pressure every morning, and thus the measurement data of each day is accumulated in the measurement data storage unit 161. The measurement data may include measurement values of systolic blood pressure and diastolic blood pressure.

The first determination unit 154 reads the measurement data from the measurement data storage unit 161 and determines, on the basis of the measurement data, whether or not the blood pressure has changed in the first time period toward achieving a predetermined goal. The first determination unit 154 uses measurement values of each day in the first time period to make a determination. For example, if the first time period is from April 1 to April 7, the measurement values of 7 days from April 1 to April 7 is used. Note that in this example, the measurement values of all of the days in the first time period are used, but no such limitation is intended. In the case in which the first time period is from April 1 to April 7, for example, the measurement values of three days of, April 1, April 4, and April 7, may be used. When the user takes a plurality of measurements in one day, an average of the plurality of measurement values obtained from the measurements may be used as the measurement value of the day for the determination. Also, a measurement value obtained from a measurement performed in a specific time period of each day (for example, from 7 o'clock to 9 o'clock) may be used for determination.

The user's goal is, for example, to reduce blood pressure below a target value. According to “Guidelines for the Management of Hypertension” developed by the Japanese Society of Hypertension, hypertension corresponds to having a systolic blood pressure measured at home of 135 mmHg or greater or a diastolic blood pressure measured at home of 85 mmHg or greater. The target value can be set according to “Guidelines for the Management of Hypertension”. In this case, the user's goal corresponds to lowering the systolic blood pressure to less than 135 mmHg and lowering the diastolic blood pressure to less than 85 mmHg.

Lowering the blood pressure to the target value can include lowering the blood pressure to the target value as well as keeping a state of the blood pressure being below the target value. In this case, the user's goal can be divided into a stage of lowering the blood pressure below the target value (hereinafter referred to as the first stage) and a stage of keeping the blood pressure below the target value (hereinafter referred to as the second stage). The first determination unit 154 determines whether the first time period corresponds to either one of the first stage and the second stage on the basis of the measurement data. In an example in which the specific time period is a time period just prior to the first time period, for example, the first determination unit 154 calculates an average value for the blood pressure over the specific time period and determines the stage to which the first time period corresponds to on the basis of the calculated blood pressure average value. For example, in the case in which the calculated blood pressure average value exceeds the target value, the first time period corresponds to the first stage, otherwise the first time period corresponds to the second stage. The specific time period may be the same as the first time period, for example. The specific time period may be the same length as the first time period or may be a different length to the first time period. As an example, when the first time period is from April 1 to April 7, the specific time period may be from March 25 to March 31. The first determination unit 154 makes a determination using different criteria depending on the stage.

In the first stage, the first determination unit 154 determines whether or not blood pressure has a declining trend in the first time period. For example, the first determination unit 154 calculates a blood pressure declining rate from each of the measurement values of the days in the first time period, and in the case in which the blood pressure declining rate is greater than a preset threshold, the first determination unit 154 determines that the blood pressure has a declining trend in the first time period, and otherwise determines that the blood pressure does not have a declining trend in the first time period. The blood pressure declining rate represents, for example, a decreasing blood pressure per day. For the blood pressure declining rate, a greater value indicates a decrease (improvement) in the blood pressure. The blood pressure declining rate can be determined, for example, by calculating a linear function that fits the measurement value using the least squares method. The threshold is, for example, 0.2 (mmHg/1 day).

In the second stage, the first determination unit 154 makes a determination on the basis of the average blood pressure value of the first time period, for example. Specifically, the first determination unit 154 calculates the average blood pressure value of the first time period on the basis of the measurement data. In the case in which the average blood pressure value is less than the target value, the first determination unit 154 determines that the blood pressure has changed in the first time period toward achieving the goal, and otherwise, the first determination unit 154 determines that the blood pressure has not changed in the first time period toward achieving the goal. Note that the first determination unit 154 may make a determination on the basis of the number of days that the blood pressure measurement value exceeded the target value. Specifically, the first determination unit 154 calculates the number of days that the blood pressure measurement value exceeded the target value on the basis of the measurement data. In the case in which the number of days the blood pressure measurement value exceeded the target value is less than a threshold, the first determination unit 154 determines that the blood pressure has changed in the first time period toward achieving the goal, and otherwise, the first determination unit 154 determines that the blood pressure has not changed in the first time period toward achieving the goal. When the first time period is seven days, the threshold is set, for example, to 2 days. Note that the first determination unit 154 may make a determination on the basis of the number of days that the blood pressure measurement value was less than the target value. In this case, when the number of days the blood pressure measurement value was less than the target value is greater than a threshold, the first determination unit 154 determines that the blood pressure has changed in the first time period toward achieving the goal, and otherwise, the first determination unit 154 determines that the blood pressure has not changed in the first time period toward achieving the goal.

Note that the goal may include only one of lowering the blood pressure to the target value and keeping the state of the blood pressure being below the target value. When the goal is to lower the blood pressure to the target value, the method described above with respect to the first stage can be employed. When the goal is to keep the state of blood pressure being below the target value, the method described above with respect to the second stage can be employed.

The behavior data acquisition unit 155 acquires behavior data relating to the medication of the user and stores the acquired behavior data in the behavior data storage unit 162. In one example, the display control unit 152 displays a confirmation screen on the display device 106 for confirming whether or not the antihypertensive agent has been taken, and the user uses the input device 107 to input whether or not the antihypertensive agent has been taken. The behavior data acquisition unit 155 receives, from the input unit 151, medication information indicating whether or not the antihypertensive agent has been taken. The confirmation screen is displayed at each time the antihypertensive agent should be taken. For example, in the case in which the antihypertensive agent should be taken twice a day after breakfast and after dinner, the display control unit 152 causes the confirmation screen to be displayed on the display device 106 at, for example, 8:00 and 22:00. In this manner, medication information is recorded at each time the antihypertensive agent should be taken.

The second determination unit 156 reads the behavior data from the behavior data storage unit 162, and determines, on the basis of the behavior data, whether or not the user has taken the medication in the second time period in a manner that satisfies a predetermined condition. The second time period includes the day prior to the first time period and at least a portion of the first time period. For example, the first time period is from April 1 to April 7, and the second time period is from March 31 to April 6. The condition is, for example, that the medication adherence exceeds a preset threshold. The second determination unit 156 calculates the medication adherence of the second time period on the basis of the behavior data. In the case in which the medication adherence is greater than the threshold, the second determination unit 156 determines that the user took the medication in the second time period in a manner that satisfies the condition, and otherwise, the second determination unit 156 determines that the user did not take the medication in the second time period in a manner that satisfies the condition. The threshold is, for example, 0.9 (90%).

The information presentation unit 157 presents to the user information (advice) that facilitates achieving the goal when the first determination unit 154 determines that the blood pressure has not changed in the first time period toward achieving the goal and the second determination unit 156 determines that the user took the medication in the second time period in a manner that satisfies the condition. The advice may be presented by the information being displayed on the display device 106, for example. Alternatively, the advice may be output via the speaker 109.

The information presentation unit 157 acquires the information to be presented to the user from the database 163. The database 163 holds information associated with a plurality of factors that cause the blood pressure to not change toward achieving the goal. Examples of factors which cause the blood pressure to not change toward achieving the goal include medication, food intake, stress, smoking, medication incompatibility, and the like. The information associated with medication may be, for example, a message saying “Please take the antihypertensive agent as instructed by the physician”. The information associated with food intake may be, for example, a message saying “Please be sure to reduce your salt intake”. The information associated with stress may be, for example, a message saying “Please refresh your body and mind with exercise or sufficient sleep”. The information associated with smoking may be, for example, a message saying “Please stop smoking”. Information associated with drug incompatibility may be, for example, a message saying “The medication may not be compatible. Please be advised to consult a physician”.

When the second determination unit 156 determines that the user has appropriately taken the medication in the second time period, the factor is determined to not be the medication. Thus, the information presentation unit 157 presents information associated with factors other than the medication to the user. When a plurality of factors are candidates, the information presentation unit 157 presents information associated with at least one of the factors. For example, the information presentation unit 157 randomly selects one of the plurality of factors and presents information associated with the selected factor. Note that the information presentation unit 157 may select one of the plurality of factors on the basis of attribute information of the user. Attribute information may include information indicating a user's physical characteristics (for example, gender, age, height, and the like), information indicating the user's habits (for example, whether or not they smoke), and the like. In the case in which the user is known to not smoke from the attribute information of the user, smoking is removed from the candidates for factors.

Note that the database 163 may be provided on the outside of the terminal device 100, for example, in a server on a communication network.

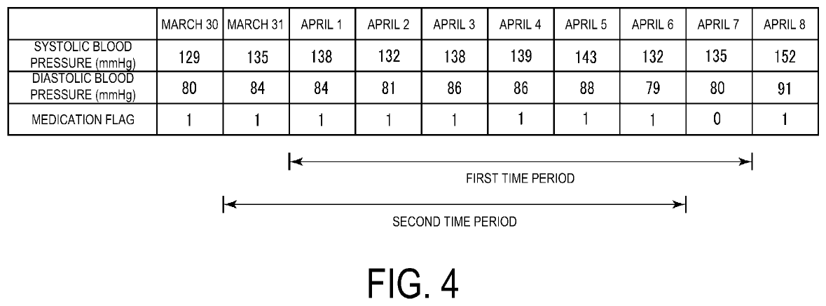

An example of determination processing by the first determination unit 154 and the second determination unit 156 will be described with reference to FIG. 4. FIG. 4 illustrates measurement data and behavior data of a time period from March 30 to April 8. In FIG. 4, the measurement data includes measurement values of the systolic blood pressure and the diastolic blood pressure obtained in the time period after waking up. The medication flag “1” indicates that the antihypertensive agent was taken, and the medication flag “0” indicates that the antihypertensive agent was not taken.

The first time period is from April 1 to April 7, the average value of the systolic blood pressure for the first time period is 136.7 (mmHg), and the average value of the diastolic blood pressure for the first time period is 83.4 (mmHg). Since the average value of the systolic blood pressure for the first time period is less than 140 (mmHg), the first determination unit 154 determines the current stage to be the second stage. Furthermore, since the average value of the diastolic blood pressure for the first time period is less than 85 (mmHg) and the average value of the systolic blood pressure for the first time period is not less than 135 (mmHg), the first determination unit 154 determines that the blood pressure did not change in the first time period toward achieving the goal.

The second time period is from March 30 to April 6, and the medication adherence in the second time period is 1. Since the medication adherence in the second time period exceeds 0.9, the second determination unit 156 determines that the user has taken the medication in the second time period in a manner that satisfies the condition.

The present embodiment is described using an example in which all the functions of the terminal device 100 are implemented by a general-purpose processor. However, some or all of the functions may be implemented by one or more dedicated processors.

### Operation Example

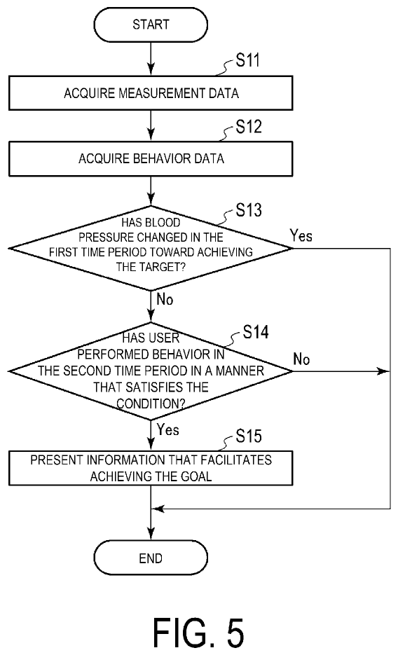

FIG. 5 illustrates an example of an operation flow when the terminal device 100 executes user health management.

In step S11 of FIG. 5, the control unit 101 of the terminal device 100 operates as the measurement data acquisition unit 153 and acquires measurement data from the blood pressure monitor. Specifically, the control unit 101 receives measurement data from the blood pressure monitor via the communication interface 110. In this example, to facilitate explanation, the measurement data includes measurement values of the systolic blood pressure but does not include measurement values of the diastolic blood pressure.

In step S12, the control unit 101 operates as the behavior data acquisition unit 155 and acquires behavior data relating to the medication of the user. For example, the control unit 101 displays a confirmation screen for confirming whether or not the antihypertensive agent has been taken, on the display device 106. When the user uses the input device 107 and inputs whether or not the antihypertensive agent has been taken, the control unit 101 acquires the medication information indicating whether or not the antihypertensive agent has been taken.

The processing illustrated in steps S11 and S12 are routinely repeated.

Accordingly, measurement data and behavior data are accumulated in the storage unit 105.

The processing sequence from step S13 to S15 is executed at a specific timing. For example, the processing sequence is executed periodically (for example, weekly). Note that the processing sequence may be executed at a timing specified by the user.

In step S13, the control unit 101 operates as the first determination unit 154 and determines, on the basis of the measurement data, whether or not the blood pressure has changed in the first time period toward achieving a predetermined goal. In the case in which the control unit 101 determines that the blood pressure has changed in the first time period toward achieving the target, the processing ends. In the case in which the control unit 101 determines that the blood pressure has not changed in the first time period toward achieving the target, the processing proceeds to step S14. For example, the control unit 101 calculates the average value of the systolic blood pressure for the first time period. In the case in which the calculated average value of the systolic blood pressure for the first time period is less than a threshold, it is determined that the blood pressure has changed in the first time period toward achieving the goal, and otherwise, it is determined that the blood pressure has not changed in the first time period toward achieving the goal.

In step S14, the control unit 101 operates as the second determination unit 156 and determines, on the basis of the behavior data, whether or not the user took medication in the second time period in a manner that satisfies the predetermined condition. In the case in which the control unit 101 determines that the user has taken the medication in the second time period in a manner that satisfies the condition, the processing proceeds to step S15, and in the case in which the control unit 101 determines that the user has not taken the medication in the second time period in a manner that satisfies the condition, the processing ends. For example, the control unit 101 calculates the medication adherence of the second time period. In the case in which the medication adherence is greater than the threshold, the control unit 101 determines that the user took the medication in the second time period in a manner that satisfies the condition, and otherwise, the control unit 101 determines that the user did not take the medication in the second time period in a manner that satisfies the condition.

In step S15, the control unit 101 operates as the information presentation unit 157 and presents, to the user, information that facilitates achieving the goal. For example, the control unit 101 may cause a message to be displayed on the display device 106 saying “Your salt intake may be too high. Please be sure to reduce your salt intake”.

Furthermore, the processing procedure illustrated in FIG. 5 is merely an example and the processing procedure can be appropriately changed. For example, in the case in which it is determined that the user did not take the medication in the second time period in a manner that satisfies the condition (No in step S14), the control unit 101 may output a message that prompts the user to take the medication without fail.

Effects

According to the terminal device 100 with the configuration described above, information that facilitates achieving the goal is presented to the user when the user has appropriately taken the antihypertensive agent in the second time period but the blood pressure has not changed in the first time period toward achieving the goal. Typically, the user measures blood pressure after waking up and then eats breakfast and takes the antihypertensive agent. In this case, the blood pressure measurement value depends largely on whether or not the user took the antihypertensive agent the day before. Thus, the blood pressure measurement value of the first day in the first time period depends largely on whether or not the user has taken the antihypertensive agent on the day prior to the first time period. Thus, the change in blood pressure in the first time period is affected not only by taking the medication in the first time period, but also by taking the medication in a time period prior to the first time period. Thus, by the second time period including the day prior to the first time period, whether or not the cause of the blood pressure not changing in the first time period toward achieving the goal is the taking of the medication can be determined with greater accuracy. This allows appropriate information that facilitates achieving the goal to be presented to the user. In other words, appropriate advice for achieving a set goal relating to the health of the user can be provided to the user. The user is able to receive advice and take action to achieve the goal.

Furthermore, the first determination unit 154, for example, makes a determination on the basis of the average value of the blood pressure for the first time period or the number of days that the blood pressure was less than the threshold in the first time period. In this way, the determination is made using the measurement values of all of the days in the first time period, allowing a determination to be made based on an actual change in the blood pressure in the first time period. As a result, appropriate advice can be provided to the user.

### Modified Examples

Note that the present invention is not limited to the embodiments described above.

In the embodiment described above, the second time period includes a day prior to the first time period. However, the second time period may not include a day prior to the first time period. For example, the second time period may be the same as the first time period. In the case in which the first time period is a long time period, such as one month, the user's behavior in the time period prior to the first time period has little effect on the indicator relating to the user in the first time period. Thus, even when the second time period does not include the day prior to the first time period, whether or not the cause of the blood pressure not changing in the first time period toward achieving the goal is the taking of the medication can be determined with accuracy. In the case in which the first time period is a short time period, such as one week, the second time period preferably includes the day prior to the first time period.

Blood pressure is an example of an indicator relating to a user, and the medication is an example of a user's behavior. The indicator relating to the user is information relating to the biological information or behavior of the user that is measurable by a sensor. Instead of blood pressure, the indicator may be heart rate, pulse rate, an electrocardiogram, pulse wave, weight, hours of sleep, and the like. Instead of medication, the user's behavior may be exercise, food intake, drinking alcohol, sleep, work, and the like. The behavior may be able to be measured by a discretionary sensor or may be non-measurable.

In one embodiment, the indicator relating to the user is weight and the user's behavior is exercise. In this embodiment, advice is presented to the user when the user's weight does not change toward achieving the goal, even though the user is sufficiently exercising. This allows the user to take action to achieve the goal.

Specifically, the control unit 101 acquires measurement data output from a weight scale and determines, on the basis of the acquired measurement data, whether or not the weight has changed in the first period toward achieving a predetermined goal. The goal may be set to reduce weight to a certain value (for example, 65 (kg)) or may be set to reduce weight by a specific value (for example, 5 (kg)). Furthermore, the control unit 101 acquires behavior data relating to the exercise of the user and determines, on the basis of the acquired behavior data, whether or not the user has exercised in the second time period in a manner that satisfies a predetermined condition. Behavior data relating to the exercise of the user includes, for example, a history of the amount of exercise. The amount of exercise includes not only exercise relating to athletic activities such as sports, but also the amount of exercise (also referred to as amount of activity) relating to life activities, such as walking and housework. The amount of exercise can be expressed, for example, as calorie consumption. The amount of exercise is an example of an indicator relating to the user's behavior and can be measured with a meter for the amount of exercise. The indicator associated with user behavior may be steps, amount of activity, and the like. In the case in which it is determined that the weight has not changed in the first time period toward achieving the goal and exercise was performed in the second time period in a manner that satisfies the condition, the control unit 101 presents to the user information associated with a factor other than weight, i.e., information that facilitates achieving the goal.

The measurement device that measures an indicator of a user's behavior may be provided in the terminal device. For example, the control unit 101 may measure the number of steps taken by the user on the basis of the output of the acceleration sensor 108. In this case, the user does not need to input the behavior data. Thus, user convenience is improved. Also, the measurement device may be provided outside the terminal device. In this case as well, the terminal device acquires the behavior data from the measurement device via wireless communication, thereby the need for the user to input the behavior data is eliminated.

The behavior data may be generated on the basis of measurement data output from a measurement device that measures an indicator relating to the user. For example, a wearable blood pressure monitor that measures blood pressure per heartbeat may be used. The blood pressure monitor can measure the user's blood pressure over 24 hours. When the user takes an antihypertensive agent, the user's blood pressure decreases in a characteristic manner corresponding to the type of antihypertensive agent and/or the physiological characteristics of the user. Such a characteristic decrease in blood pressure can be detected from the blood pressure waveform obtained by the blood pressure monitor, for example, by using a model generated by machine learning. The control unit 101 determines whether or not a characteristic variation in the blood pressure due to the user taking the antihypertensive agent is included in the blood pressure waveform in a preset time period (for example, from 7:00 to 9:00). In this way, the control unit 101 can detect, on the basis of the measurement data generated by the blood pressure monitor, that the user has taken the antihypertensive agent. As a result, the user does not need to input information indicating whether or not the antihypertensive agent has been taken, and user convenience is improved. Note that in the case in which the control unit 101 determines that a characteristic variation in the blood pressure is not included in the blood pressure waveform of the preset time period, the control unit 101 may display a confirmation screen for confirming whether or not the antihypertensive agent has been taken, on the display device 106.

In the embodiments described above, the medication is the medication to be taken (oral medication). However, the medication is not limited to medication to be taken and may be medication for pasting, medication for applying, medication used for radiation, a self-injected medication, or the like. The term “use” when used to describe using a medication means orally taking, pasting, applying, radiation, injection, and the like.

In the above-described embodiments, the terminal device functioning as a health management device is a separate device from the measurement device that measures an indicator relating to a user. However, the health management device can be provided in the measurement device.

In short, the present invention is not limited to the embodiment described above as is, and the components can be modified and embodied within a range that does not depart from the gist in a stage of implementation. Further, various inventions can be formed by appropriately combining a plurality of constituent elements disclosed in the embodiment described above. For example, some constituent elements may be omitted from the entire constituent elements shown in the embodiment. Furthermore, the constituent elements of different embodiments may be combined appropriately.

### Supplementary Notes

A part or the entirety of the embodiment can be described, as described in the following supplementary notes in addition to the scope of the claims, but the present invention is not limited thereto.

### (Supplementary Note 1)

A health management device (20) includes a measurement data acquisition unit (21) configured to acquire measurement data output from a measurement device configured to measure an indicator relating to a user, a first determination unit (22) configured to determine, on the basis of the measurement data, whether or not the indicator has changed in a first time period toward achieving a preset goal, a behavior data acquisition unit (23) configured to acquire behavior data relating to behavior of the user, a second determination unit (24) configured to determine, on the basis of the behavior data, whether or not the user has performed the behavior in a second time period in a manner that satisfies a preset condition, the second time period including at least a portion of the first time period, and an information presentation unit (25) configured to present information that facilitates achieving the goal in a case in which the first determination unit determines that the indicator has not changed in the first time period toward achieving the goal and the second determination unit determines that the user has performed the behavior in the second time period in a manner that satisfies the condition.

### REFERENCE SIGNS LIST

10 Health management system
20 Terminal device
21 Measurement data acquisition unit
22 First determination unit
23 Behavior data acquisition unit
24 Second determination unit
25 Information presentation unit
30 Blood pressure monitor
100 Terminal device
101 Control unit
102 CPU
103 RAM
104 ROM
105 Storage unit
106 Display device
107 Input device
108 Acceleration sensor
109 Speaker
110 Communication interface
111 Battery
151 Input unit
152 Display control unit
153 Measurement data acquisition unit
154 First determination unit
155 Behavior data acquisition unit
156 Second determination unit
157 Information presentation unit
161 Measurement data storage unit
162 Behavior data storage unit
163 Database

1. A health management device, comprising:
one or more processors configured to:
acquire measurement data output from a measurement device configured to measure an indicator relating to biological information of a user;
determine, on the basis of the measurement data, whether or not the indicator has changed in a first time period toward achieving a preset goal;
generate behavior data relating to behavior of the user on the basis of the measurement data;
determine, on the basis of the behavior data, whether or not the user has performed the behavior in a second time period in a manner that satisfies a preset condition, the second time period including at least a portion of the first time period; and
present information that facilitates achieving the goal in a case in which it is determined that the indicator has not changed in the first time period toward achieving the goal and is determined that the user has performed the behavior in the second time period in a manner that satisfies the condition.

2. The health management device according to claim 1, wherein the second time period further includes a day prior to the first time period.

3. The health management device according to claim 1, wherein the one or more processors are further configured to calculate an average value of the indicator for the first time period on the basis of the measurement data and determine, on the basis of a comparison between the average value being calculated and a threshold that is preset, whether or not the indicator has changed in the first time period toward achieving the goal.

4. The health management device according to claim 1, wherein the one or more processors are further configured to calculate, on the basis of the measurement data, a number of days that the indicator was greater or less than a preset first threshold, in the first time period, and determine, on the basis of a comparison between the number of days being calculated and a preset second threshold, whether or not the indicator has changed in the first time period toward achieving the goal.

5. The health management device according to claim 1, wherein the indicator is blood pressure, and the behavior is use of medication.

6. The health management device according to claim 1, wherein
the measurement device is a blood pressure monitor configured to measure blood pressure per heartbeat, and
the one or more processors are further configured to generate the behavior data on the basis of the measurement data output from the blood pressure monitor.

7. A health management method executed by a health management device, the method comprising:
acquiring measurement data output from a measurement device configured to measure an indicator relating to biological information of a user;
determining, on the basis of the measurement data, whether or not the indicator has changed in a first time period toward achieving a preset goal;
generating behavior data relating to behavior of the user on the basis of the measurement data;
determining, on the basis of the behavior data, whether or not the user has performed the behavior in a second time period in a manner that satisfies a preset condition, the second time period including at least a portion of the first time period; and
presenting information that facilitates achieving the goal in a case in which it is determined that the indicator has not changed in the first time period toward achieving the goal and it is determined that the user has performed the behavior in the second time period in a manner that satisfies the condition.

8. A non-transitory recording medium storing a program for causing a computer to perform functions in the health management device according to claim 1.
